(19) United States
(12) Patent Application Publication
(10) Pub. No.: US 2016/0113530 A1
(43) Pub. Date: Apr. 28, 2016

(54) METHOD AND APPARATUS FOR ACQUIRING BIOLOGICAL INFORMATION, AND WRIST WATCH-TYPE TERMINAL USING THE SAME

(71) Applicant: SAMSUNG ELECTRONICS CO., LTD., Suwon-si (KR)

(72) Inventors: TAKESHI NAGAHIRO, Kanagawa (JP); Takahiro TOKUMIYA, Kanagawa (JP); Naoyuki TAKADA, Kanagawa (JP); Yosuke AOYAGI, Kanagawa (JP)

(73) Assignee: SAMSUNG ELECTRONICS CO., LTD., Suwon-si (KR)

(21) Appl. No.: 14/921,543

(22) Filed: Oct. 23, 2015

(30) Foreign Application Priority Data
Oct. 23, 2014 (JP) 2014-215975
Jan. 30, 2015 (KR) 10-2015-0015331

(51) Int. Cl. A61B 5/021; A61B 5/00

(52) U.S. Cl. CPC A61B 5/02125; A61B 5/0075; A61B 5/721; A61B 5/0082; A61B 5/681; A61B 5/6843

(57) ABSTRACT

A method and an apparatus for acquiring biological information, and a wrist watch-type terminal using the same, are provided. The apparatus includes a first detector configured to irradiate a living body with a first light of a first wavelength, and detect a second light that is reflected from the living body, and a second detector configured to irradiate the living body with a third light of a second wavelength, and detect a fourth light that is reflected from the living body. The apparatus further includes a processor configured to determine a pulse wave of the living body based on a subtraction value of subtracting a detection signal that is determined by irradiating the third light of the second wavelength from a detection signal that is determined by irradiating the first light of the first wavelength.

### CROSS-REFERENCE TO RELATED APPLICATION

This application claims priority from Japanese Patent Application No. 2014-215975, filed on Oct. 23, 2014, in the Japanese Patent Office, and Korean Patent Application No. 10-2015-0015331, filed on Jan. 30, 2015, in the Korean Intellectual Property Office, the disclosures of which are incorporated herein in their entireties by reference.

### BACKGROUND

1. Field

Methods and apparatuses consistent with exemplary embodiments relate to methods and apparatuses for acquiring biological information, and wrist watch-type terminals using the same.

2. Description of the Related Art

When light of a predetermined wavelength is irradiated to a living body, the light is scattered (reflected or transmitted) in an endodermis, a dermis surface, peripheral blood vessels, fat, and arteries of the living body. Corresponding periodic movements may be observed by reflected light obtained as a pulse of a blood vessel periodically moves within a predetermined time or by transmitted light penetrating the living body. Therefore, a pulse wave is measured by analyzing the reflected light or the transmitted light.

In this regard, it is noted that Japanese Unexamined Patent Application Publication No. 2002-369805 discloses a technique of measuring a pulse wave by subtracting a detection signal of reflected light quantity on a skin surface obtained by irradiating a living body with light having a wavelength shorter than that of a near-infrared light from a detection signal of reflected light quantity on a blood vessel, which is obtained by irradiating the living body with the near-infrared light.

The technique disclosed in Japanese Unexamined Patent Application Publication No. 2002-369805 is studied to remove a noise due to movement of the living body; however, the noise due to movement of the living body is only 4% to 5% of the entire noise added to the pulse wave. Therefore, it is difficult to greatly improve measurement accuracy of the pulse wave even if the noise due to movement of the living body is removed.

### SUMMARY

Exemplary embodiments address at least the above problems and/or disadvantages and other disadvantages not described above. Also, the exemplary embodiments are not required to overcome the disadvantages described above, and may not overcome any of the problems described above.

One or more exemplary embodiments provide methods and apparatuses for acquiring biological information, and wrist watch-type terminals using the same, which are capable of precisely measuring a pulse wave.

According to an aspect of an exemplary embodiment, there is provided an apparatus for acquiring biological information, the apparatus including a first detector configured to irradiate a living body with a first light of a first wavelength, and detect a second light that is reflected from or transmitted in the living body by irradiating the first light of the first wavelength, and a second detector configured to irradiate a living body with a third light of a second wavelength, and detect a fourth light that is reflected from or transmitted in the living body by irradiating the third light of the second wavelength. The apparatus further includes a processor configured to determine a pulse wave of the living body based on a subtraction value of subtracting a detection signal that is obtained by detecting the fourth light from a detection signal that is obtained by detecting the second light.

The first light of the first wavelength may be a red or infrared light.

The third light of the second wavelength may be a green light.

The first light of the first wavelength may be scattered (reflected or transmitted) in a blood vessel disposed deeper than a dermis of the living body, and the third light of the second wavelength may be scattered (reflected or transmitted) near the dermis.

The first detector may include a first light source configured to emit the first light of the first wavelength, and a first light receiver configured to detect the second light reflected from or transmitted in the living body, the second detector may include a second light source configured to emit the third light of the second wavelength, and a second light receiver configured to detect the fourth light reflected from or transmitted in the living body, and a distance between the first light source and the first light receiver is farther than a distance between the second light source and the second light receiver.

The second light receiver may be shared with the first light receiver.

The irradiation of the living body with the first light of the first wavelength and the irradiation of the living body with the third light of the second wavelength may be performed during a period so that irradiation timings thereof are not overlapped.

The period may be substantially equal to a period of the pulse wave of the living body.

According to an aspect of another exemplary embodiment, there is provided a wrist watch-type terminal including a biological information acquisition apparatus, the biological information acquisition apparatus including a first detector configured to irradiate a living body with a first light of a first wavelength, and detect a second light that is reflected from or transmitted in the living body by irradiating the first light of the first wavelength, and a second detector configured to irradiate the living body with a third light of a second wavelength, and detect a fourth light that is reflected from or transmitted in the living body by irradiating the first light of the first wavelength. The apparatus further includes a processor configured to determine a pulse wave of the living body based on a subtraction value of subtracting a detection signal that is obtained by detecting the fourth light from a detection signal that is obtained by detecting the second light.

According to an aspect of another exemplary embodiment, there is provided a method of acquiring biological information, the method including irradiating a living body with a first light of a first wavelength, detecting a second light that is reflected from or transmitted in the living body by irradiating the first light of the first wavelength, irradiating the living body with a third light of a second wavelength, and detecting a fourth light that is reflected from or transmitted in the living body by irradiating the third light of the second wavelength. The method further includes determining a pulse wave of the living body based on a subtraction value of subtracting a detection signal that is obtained by detecting the fourth light from a detection signal that is obtained by detecting the second light.

The first light of the first wavelength may be a red or infrared light.

The third light of the second wavelength may be a green light.

The first light of the first wavelength may be scattered (reflected or transmitted) in a blood vessel disposed deeper than a dermis of the living body, and the third light of the second wavelength may be scattered (reflected or transmitted) near the dermis.

The irradiation of the living body with the first light of the first wavelength and the irradiation of the living body with the third light of the second wavelength may be performed during a period so that irradiation timings thereof are not overlapped.

The period may be substantially equal to a period of the pulse wave of the living body.

According to an aspect of another exemplary embodiment, there is provided an apparatus for acquiring biological information, the apparatus including a substrate, a light source disposed on the substrate, the light source being configured to emit a light to a living body, and a light receiver disposed on the substrate, the light receiver being configured to receive a light that is reflected from or transmitted in the living body. The apparatus further includes a processor configured to determine a pulse wave of the living body based on a detection signal of the received light, and one of the light source and the light receiver may be disposed around another one of the light source and the light receiver.

The one of the light source and the light receiver may respectively include light sources or light receivers disposed on two or more approximately concentric circles, and the processor may be further configured to select one of the light sources or the light receivers satisfying a condition, and determine the detection signal of the light that is received by a light receiver corresponding to the selected one of the light sources or the light receivers.

The one of the light source and the light receiver may respectively include light sources or light receivers disposed on an approximately straight line, and the processor may be further configured to select one of the light sources or the light receivers satisfying a condition, and determine the detection signal of the light that is received by a light receiver corresponding to the selected one of the light sources or the light receivers.

The one of the light source and the light receiver may respectively include light sources or light receivers disposed on a spiral centering around the other one of the light source and the light receiver, and the processor may be further configured to select one of the light sources or the light receivers satisfying a condition, and determine the detection signal of the light that is received by a light receiver corresponding to the selected one of the light sources or the light receivers.

The one of the light source and the light receiver may respectively include light sources or light receivers that are ring-shaped, and the processor may be further configured to select one of the light sources or the light receivers satisfying a condition, and determine the detection signal of the light that is received by a light receiver corresponding to the selected one of the light sources or the light receivers.

The apparatus may further include a pressure detector configured to detect a contact pressure of the light source or the light receiver on the living body, and the processor may be further configured to select the light source or the light receiver contacting the living body with the contact pressure corresponding to a pressure, and determine the detection signal of the light that is received by a light receiver corresponding to the selected light source or light receiver.

The apparatus may further include a pressure detector configured to detect a contact pressure of the light source or the light receiver on the living body, the light source or the light receiver being disposed on an approximately concentric circle, and a pressure adjuster configured to adjust the contact pressure on the living body to a pressure.

The processor may be further configured to determine a pulse wave in a different position of the living body by operating the other one of the light source and the light receiver at a different distance from the one of the light source and the light receiver, and determine a blood pressure of the living body based on a propagation time of the determined pulse wave.

A wavelength of the light emitted to the living body may change based on a distance between the light source and the light receiver that respectively are on different sides.

The substrate may be flexible.

In response to a state of bending the flexible substrate as the biological information acquisition apparatus is attached onto the living body, the one of the light source and the light receiver may be disposed on an approximately concentric ellipsoid centering around the other one of the light source and the light receiver, and in response to a state of flattening the flexible substrate, the one of the light source and the light receiver may be disposed on an approximately concentric circle centering around the other one of the light source and the light receiver.

The light source may include a first light source configured to emit a light of a first wavelength, and a second light source configured to emit a light of a second wavelength, and the processor may be further configured to determine the pulse wave based on a subtraction value of subtracting a detection signal that is determined by irradiating the light of the second wavelength from a detection signal that is determined by irradiating the light of the first wavelength.

According to an aspect of another exemplary embodiment, there is provided a wrist watch-type terminal including a biological information acquisition apparatus, the biological information acquisition apparatus including a substrate, a light source disposed on the substrate, the light source being configured to emit a light to a living body, a light receiver disposed on the substrate, and the light receiver being configured to receive a light that is reflected from or transmitted in the living body. The apparatus further includes a processor configured to determine a pulse wave of the living body based on a detection signal of the received light, and one of the light source and the light receiver is disposed around another one of the light source and the light receiver.

### BRIEF DESCRIPTION OF THE DRAWINGS

The above and/or other aspects will be more apparent by describing exemplary embodiments, with reference to the accompanying drawings, in which:

FIG. 1 is a schematic block diagram of a biological information acquisition apparatus according to an exemplary embodiment;

FIG. 2 is a schematic plan view of a sensor of a biological information acquisition apparatus according to an exemplary embodiment;

FIG. 3 is a schematic view illustrating light emitted from a light source of a biological information acquisition apparatus and received by a light receiver through a living body according to an exemplary embodiment;

FIG. 4 is a flowchart illustrating a biological information acquisition method according to an exemplary embodiment;

FIG. 5 is a graph illustrating a light-emitting timing of a red or infrared light and a light-emitting timing of a green light according to an exemplary embodiment;

FIG. 6 is a graph illustrating a light-emitting timing of a red or infrared light and a light-emitting timing of a green light according to another exemplary embodiment;

FIG. 7 is a graph illustrating a detection signal of a first detector input to a processor according to an exemplary embodiment;

FIGS. 8A and 8B are graphs illustrating a waveform of a subtraction value resulting from subtracting a detection signal of a second detector from a detection signal of a first detector, the subtraction value, and a feature point extracted from the subtraction value according to an exemplary embodiment;

FIGS. 9A and 9B are graphs illustrating calculating of a blood pressure of a living body according to the extracted feature point of FIGS. 8A and 8B, and a regression line;

FIG. 10 is a schematic view of a sensor according to another exemplary embodiment;

FIG. 11 is a schematic view of a sensor according to another exemplary embodiment;

FIG. 12 is a schematic view of a sensor according to another exemplary embodiment;

FIG. 13 is a schematic view of a sensor according to another exemplary embodiment;

FIG. 14 is a schematic view of a sensor according to another exemplary embodiment;

FIG. 15 is a schematic view of a sensor configured to be able to detect a contact pressure of a first light source on a living body according to another exemplary embodiment;

FIG. 16 is a schematic view of a configuration capable of adjusting a contact pressure of a first light source on a living body to a predetermined value according to another exemplary embodiment;

FIG. 17 is a schematic view of a wrist watch-type terminal according to another exemplary embodiment; and

FIG. 18 is a view of a back of the wrist watch-type terminal of FIG. 17.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions may not be described in detail because they would obscure the description with unnecessary detail.

It will be understood that the terms “comprises” and/or “comprising” used herein specify the presence of stated features or components, but do not preclude the presence or addition of one or more other features or components. In addition, the terms such as “unit”, “-er (-or)”, and “module” described in the specification refer to an element for performing at least one function or operation, and may be implemented in hardware, software, or the combination of hardware and software.

First, a method and apparatus for acquiring biological information according to an exemplary embodiment will be schematically described. According to the method and apparatus for acquiring biological information according to an exemplary embodiment, a pulse wave of a living body is obtained according to a subtraction value resulting from subtracting a detection signal of a scattered light (a reflected light or a transmitted light) near a dermis generating a large amount of noise, which is obtained by irradiating light of a second wavelength from a detection signal that is an output waveform of a scattered light (a reflected light or a transmitted light) of a blood vessel, which is obtained by irradiating the living body with light of a first wavelength. Therefore, it is possible to obtain a detection signal having less noise and measure a pulse wave of a living body, and furthermore, a blood pressure of a living body with an improved level of measurement accuracy.

First, a biological information acquisition apparatus according to an exemplary embodiment will now be described in detail.

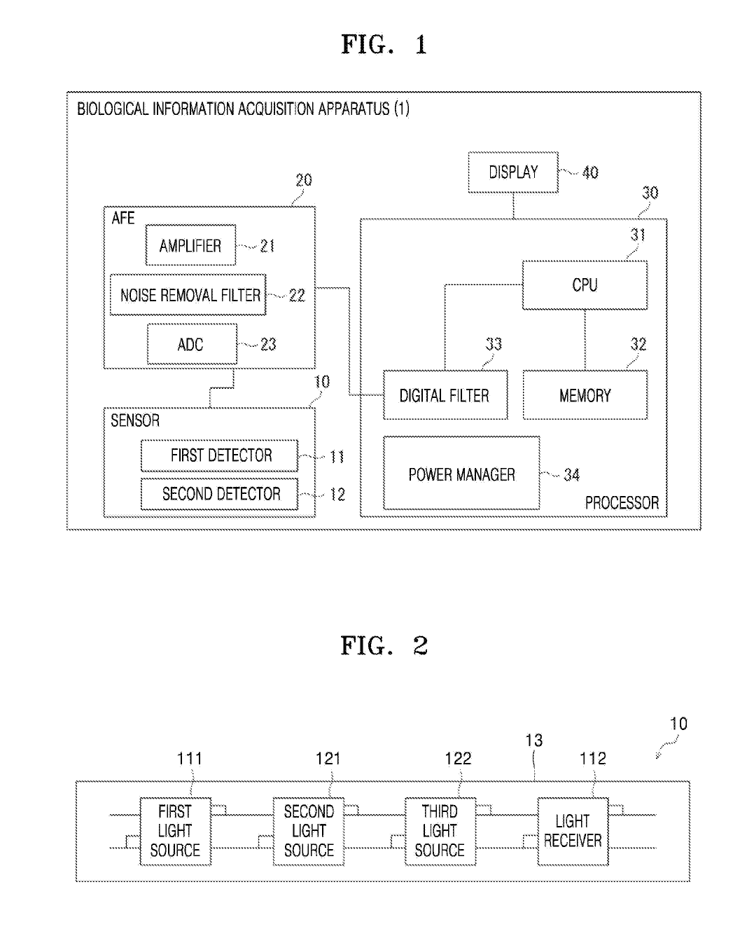

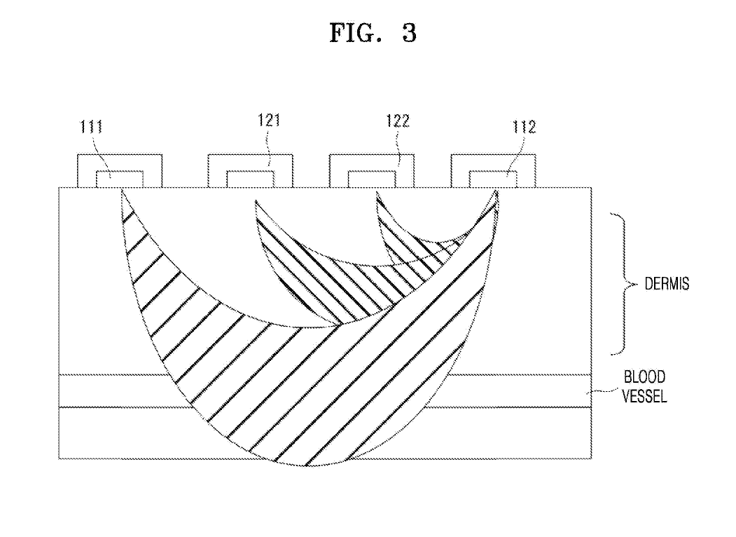

FIG. 1 is a schematic block diagram of a biological information acquisition apparatus according to an exemplary embodiment. FIG. 2 is a schematic plan view of a sensor of a biological information acquisition apparatus according to an exemplary embodiment. FIG. 3 is a schematic view illustrating light emitted from a light source of a biological information acquisition apparatus and received by a light receiver through a living body according to an exemplary embodiment.

A biological information acquisition apparatus 1 is installed in, for example, a wearable terminal and attached onto a part of a body, such as a wrist. As illustrated in FIG. 1, the biological information acquisition apparatus 1 includes a sensor 10, an analog front end (AFE) 20, a processor 30, and a display 40.

The sensor 10 includes a first detector 11 and a second detector 12 and operates according to a control signal from the processor 30. The first detector 11 may include a light source emitting a red light (for example, a wavelength in a range of 620 nm or more to 780 nm or less) or an infrared light (IR; for example, a wavelength in a range of 780 nm or more to 1100 nm or less) as a detection light, and a light receiver receiving a reflected light or transmitted light in the living body resulting from the detection light.

The second detector 12 may include a light source emitting a green light (for example, a wavelength in a range of 495 nm or more to 570 nm or less) as a detection light, and a light receiver receiving a reflected light or transmitted light in the living body resulting from the detection light.

For example, a light-emitting element such as a light-emitting diode (LED) or a laser diode (LD) may be adopted as the light source of the first detector 11 and the second detector 12. A light receiving element, for example, a charge coupled device (CCD) or a complementary metal oxide semiconductor (CMOS) image sensor may be used as the light receiver of the first detector 11 and the second detector 12. The light receiver may photoelectrically convert the received light and output a signal representing an intensity of the received light to the AFE 20.

The sensor 10 according to an exemplary embodiment receives a reflected light reflected from a blood vessel located deeper than a dermis by irradiating a living body with a red or infrared light through the first detector 11, and receives a reflected light reflected from a peripheral blood vessel near the dermis or fat by irradiating a living body with a green light through the second detector 12.

In detail, the sensor 10, as illustrated in FIG. 2, includes a first light source 111 emitting a red or infrared light as a detection light, a second light source 121 emitting a green light as a detection light, a third light source 122 emitting a green light as a detection light, and a light receiver 112 on a common substrate 13. In other words, the sensor 10 according to an exemplary embodiment includes one of the first detectors 11 and two of the second detectors 12, and the light receiver 112 of the first detector 11 is commonly used as a light receiver of two of the second detectors 12. Accordingly, the sensor 10 may be miniaturized by reducing the number of the light receiver 112.

A distance between a light receiver and a light source corresponds to how deeply light reaches into a living body. In an exemplary embodiment, as illustrated in FIG. 3, the third light source 122, the second light source 121, and the first light source 111 are arranged in an order and in a direction away from the light receiver 112. In other words, the first light source 111 for obtaining a reflected light of a blood vessel existing in a living body deeper than a dermis is arranged at a position away from the light receiver 112 in comparison with the second light source 121 and the third light source 122. Therefore, the reflected light of the blood vessel may be appropriately received. In addition, hatched portions of FIG. 3 represent a light irradiation region. Furthermore, intervals of the first to third light sources 111 to 122 may be appropriately set based on a wavelength of an emitted light.

Meanwhile, the first to third light sources 111 to 122 according to an exemplary embodiment, as illustrated in FIG. 2, are arranged on a straight line at approximately equal intervals. In the specification, the straight line may include not just a straight line in a strict sense but also an approximately straight line as will be understood by those of ordinary skill in the art. Therefore, it is possible to arrange the first to third light sources 111 to 122 on an approximately identical blood vessel when the biological information acquisition apparatus 1 is attached onto a living body, and thus measurement accuracy of a pulse wave may be improved.

The AFE 20 includes an amplifier 21, a noise removal filter 22, and an analog digital converter (ADC) 23. The amplifier 21 amplifies a detection signal from the sensor 10. The noise removal filter 22 is an analog filter and removes noise of the detection signal amplified in the amplifier 21 by analog processing. For example, the noise removal filter 22 may be an inductor capacitor (LC) filter such as a low-pass filter or a high-pass filter.

The ADC 23 may convert the detection signal from which noise is removed by the noise removal filter 22 into a digital signal. Furthermore, the ADC 23 may output the digital signal converted from the detection signal to the processor 30. The ADC 23 may output a digital value sampled at a predetermined sampling period as a detection signal.

The processor 30, for example, may be a micro computer, and includes a central processing unit (CPU) 31, a memory 32, a digital filter 33, and a power manager 34. The memory 32 may store a predetermined program. The CPU 31 may read and execute the program stored in the memory 32. The processor 30 may calculate blood pressures (a systolic blood pressure (SBP) and a diastolic blood pressure (DBP) and output a signal representing the calculated blood pressures to the display 40, based on the detection signal from the AFE 20. Moreover, the processor 30 may calculate a health index other than a blood pressure, for example, an arteriosclerosis index (AI) value.

The digital filter 33 subtracts a detection signal obtained by irradiating a living body with the detection light from the second light source 121 or the third light source 122, from a detection signal obtained by irradiating a living body with the detection light from the first light source 111. In this case, as a pulse wave corresponding to a pulse of a blood vessel repeatedly appears in the detection signal, a pulse wave of a living body may be obtained based on the detection signal.

The power manager 34 may control power supplied to the sensor 10. For example, the power manager 34 supplies a predetermined operating current to each of the first to third light sources 111 to 122 and emits the first to third light sources 111 to 122 with a predetermined intensity. Furthermore, the power manager 34 controls a supply timing of current to each of the first to third light sources 111 to 122 and intermittently emits each of the first to third light sources 111 to 122 in a predetermined timing order.

The display 40 may display information such as a pulse wave of a living body or a blood pressure indicated by a signal from the processor 30. The display 40 may include, for example, a liquid crystal display (LCD) or an electro luminescence (EL) display.

Next, a biological information acquisition method performed by the biological information acquisition apparatus 1 will now be described. The biological information acquisition method according to an exemplary embodiment involves calculating a blood pressure according to a pulse wave of a living body.

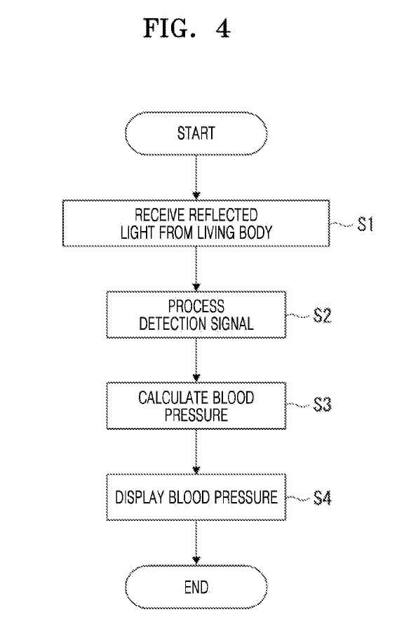

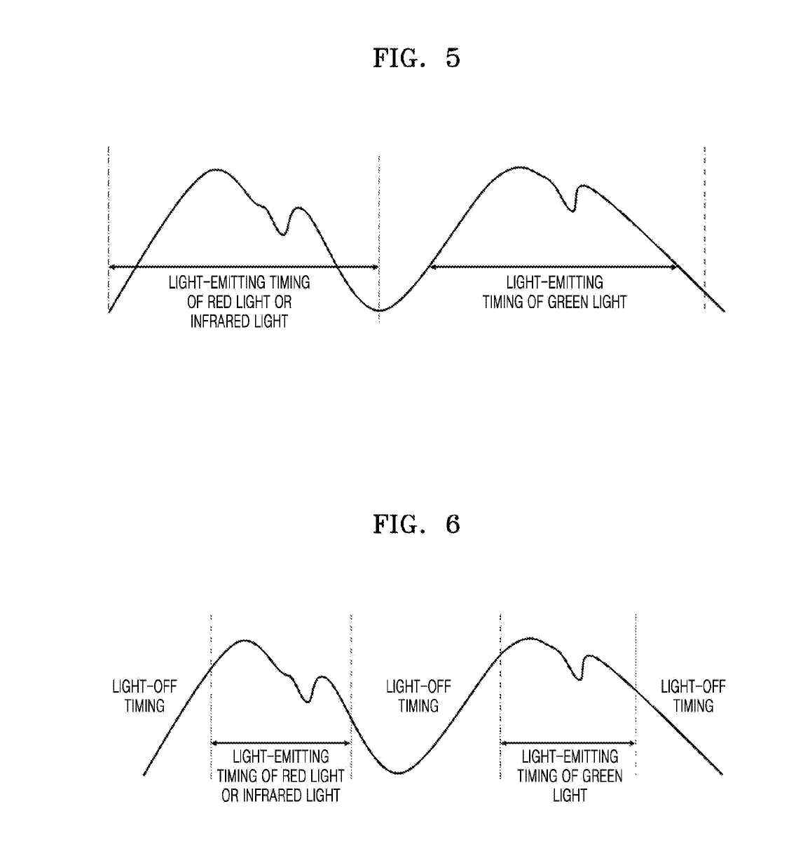

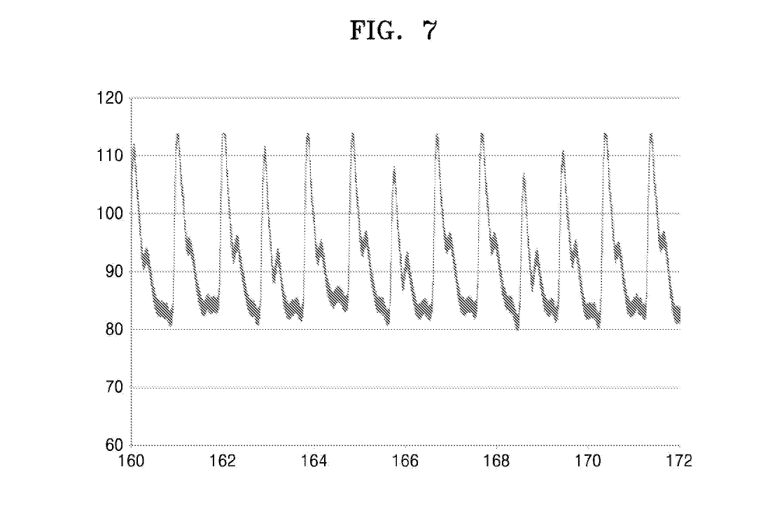

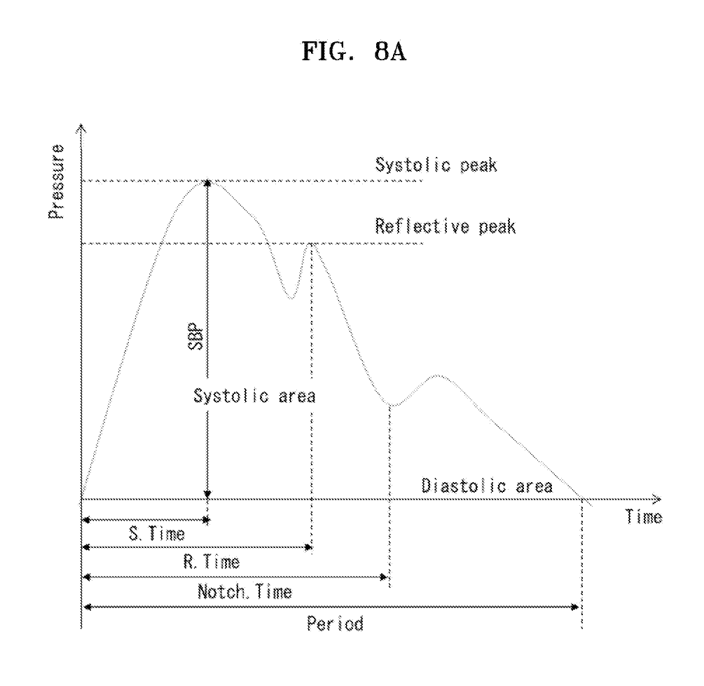

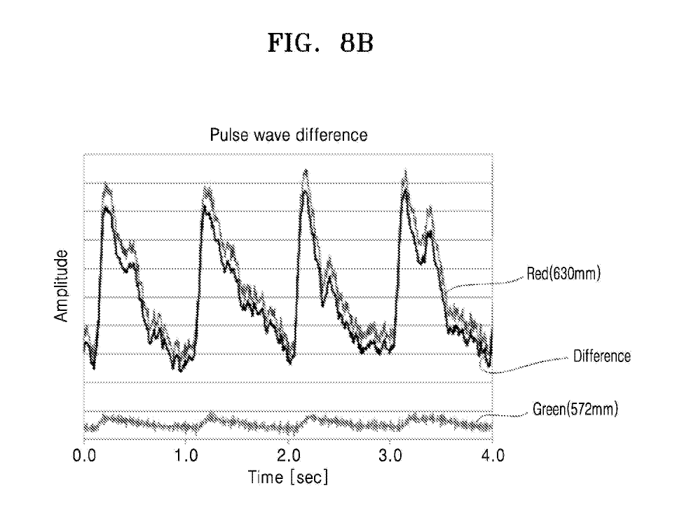

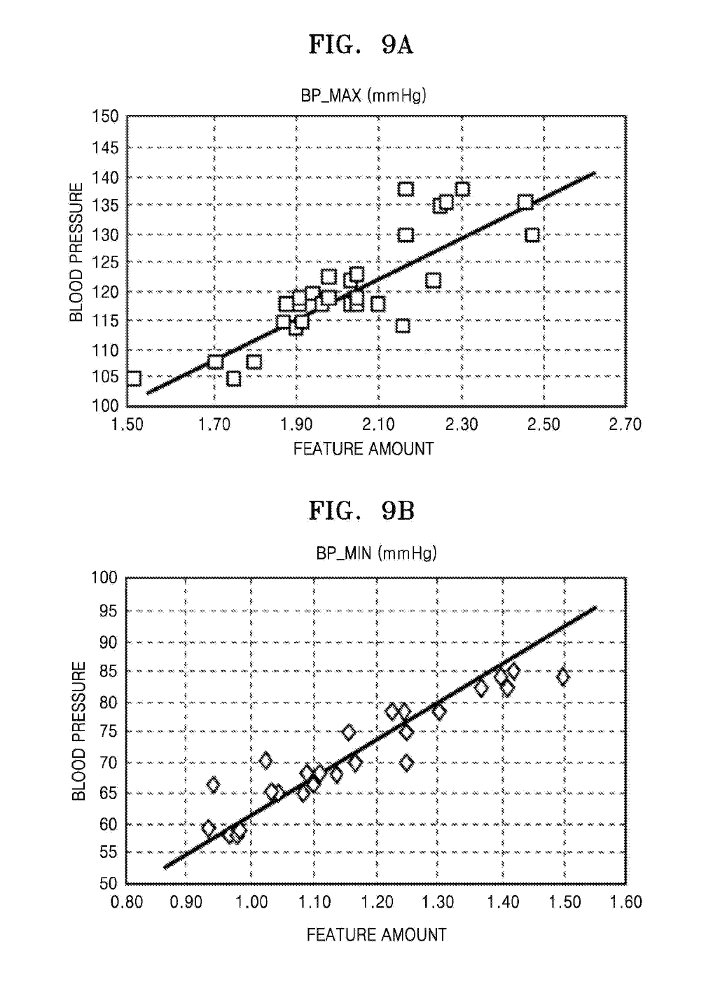

FIG. 4 is a flowchart illustrating a biological information acquisition method according to an exemplary embodiment. FIG. 5 is a graph illustrating a light-emitting timing of a red or infrared light and a light-emitting timing of a green light according to an exemplary embodiment. FIG. 6 is a graph illustrating a light-emitting timing of a red or infrared light and a light-emitting timing of a green light according to an exemplary embodiment. FIG. 7 is a graph illustrating a detection signal of a first detector input to a processor according to an exemplary embodiment. FIGS. 8A and 8B are graphs illustrating a waveform of a subtraction value resulting from subtracting a detection signal of a second detector from a detection signal of a first detector, the subtraction value, and a feature point extracted from the subtraction value according to an exemplary embodiment. FIGS. 9A and 9B are graphs illustrating calculating of a blood pressure of a living body according to the extracted feature point of FIGS. 8A and 8B, and a regression line.

First, the biological information acquisition apparatus 1 is attached onto a wrist of a living body by, i.e., a band, and as illustrated in FIG. 4, in operation 51, each detection light of the first to third light sources 111 to 122 is irradiated to the living body such that the light receiver 112 receives a reflected light from the living body.

In detail, to not overlap light-emitting timings of the first to third light sources 111 to 122, the first to third light sources 111 to 122 emit the detection light at predetermined periods and predetermined times based on a control signal from the processor 30.

In an exemplary embodiment, after sampling the pulse wave of the living body in advance, as illustrated in FIG. 5, the first light source 111 emits light within the period of approximately one cycle of the pulse wave, and the second light source 121 emits light within the period of approximately one cycle of the same pulse wave afterwards. Furthermore, the third light source 122 emits light within the period of approximately one cycle of a corresponding pulse wave.

That is, the first to third light sources 111 to 122 irradiate the living body with light in order at every period of approximately one cycle. Therefore, the detection light from each of the first to third light sources 111 to 122 may be irradiated to the living body from when respective strengths of detection signals become approximately the same during an approximately same period.

A starting point in the period of one cycle according to an exemplary embodiment may be a minimum point of the pulse wave but is not limited thereto. Furthermore, in an exemplary embodiment, the detection light from each of the first to third light sources 111 to 122 is irradiated at the entire period of one cycle, but as illustrated in FIG. 6, for example, the detection lights from the first to third light sources 111 to 122 may be intermittently emitted during approximately the same period and at the same time as those of the pulse wave. Therefore, power consumption of the first to third light sources 111 to 122 may be prevented from occurring. Furthermore, a light-emitting sequence of the light sources is not particularly limited. Furthermore, the light-emitting of the first to third light sources 111 to 122 forms a pair, but the exemplary embodiments are not limited thereto.

As described above, the red light or the infrared light is reflected from a blood vessel, and the green light is reflected from a peripheral blood vessel near a dermis or fat. Therefore, as illustrated in FIG. 3, the detection light irradiating the living body from the first light source 111 is reflected from the blood vessel, and the reflected light is received by the light receiver 112. Furthermore, the detection light irradiating the living body from the second light source 121 is reflected from the peripheral blood vessel near the dermis or the fat, and the reflected light is received by the light receiver 112. Moreover, the detection light irradiating the living body from the third light source 122 is reflected from the peripheral blood vessel near the dermis or the fat, and the reflected light is received by the light receiver 112. The light receiver 112 photoelectrically converts a signal representing an intensity of the received light, and outputs a photoelectrically converted analog signal to the AFE 20.

Referring again to FIG. 4, in operation S2, the AFE 20 processes a detection signal input from the sensor 10. In detail, the amplifier 21 of the AFE 20 amplifies the detection signal input from the sensor 10 based on a control signal of the processor 30. Furthermore, the noise removal filter 22 of the AFE 20 removes noise by filtering the amplified detection signal based on the control signal of the processor 30. Moreover, the ADC 23 of the AFE 20 digitizes the detection signal from which noise is removed based on the control signal of the processor 30, and outputs the digitized detection signal is removed to the processor 30. For example, the ADC 23 of the AFE 20 outputs a detection signal of the first detector 11 having a waveform as illustrated in FIG. 7 to the processor 30.

Referring again to FIG. 4, in operation S3, the processor 30 calculates a blood pressure according to a detection signal input from the AFE 20. As the peripheral blood vessel near the dermis or the fat exists from skin to the blood vessel, noise based on the reflected light reflected by the peripheral blood vessel or the fat is included in a detection signal of a reflected light obtained by irradiating the living body with the detection light from the first light source 111.

Therefore, the digital filter 33 of the processor 30 obtains the pulse wave of the living body according to a subtraction value resulting from subtracting a detection signal of a reflected light obtained by irradiating the living body with a detection light from the second light source 121 or the third light source 122, from the detection signal of the reflected light obtained by irradiating the living body with the detection light from the first light source 111. Furthermore, the digital filter 33 outputs a detection signal representing the subtraction value (that is, the pulse wave of the living body) to the CPU 31.

Therefore, it is possible to obtain a detection signal in which the noise caused by the detection light reflected by the peripheral blood vessel near the dermis or the fat is appropriately removed, and thus, measurement accuracy of the pulse wave of the living body may be improved.

Meanwhile, the process of subtracting the detection signal of the reflected light obtained by irradiating the living body with the detection light from the second light source 121 or the third light source 122 from the detection signal of the reflected light obtained by irradiating the living body with the detection light from the first light source 111 may reduce a load of arithmetic processing on the processor 30.

In an exemplary embodiment, the detection signal of higher intensity is selected from the detection signal of the reflected light obtained by irradiating the living body with the detection light from the second light source 121 and the detection signal of the reflected light obtained by irradiating the living body with the detection light from the third light source 122, and the selected detection signal is subtracted from the detection signal of the reflected light obtained by irradiating the living body with the detection light from the first light source 111. Therefore, the pulse wave of the living body may be measured with higher accuracy.

Next, the CPU 31 of the processor 30 calculates a blood pressure according to a detection signal input from the digital filter 33. In detail, as illustrated in FIGS. 8A and 8B, the CPU 31 identifies a period of one cycle of the pulse wave from when the subtraction value resulting from subtracting the detection signal of the reflected light obtained by irradiating the living body with the detection light from the second light source 121 or the third light source 122, from the detection signal of the reflected light obtained by irradiating the living body with the detection light from the first light source 111, becomes a positive value. In the following descriptions, the pulse wave corresponding to the period of one cycle may be referred to as one pulse wave. The CPU 31 sets a timing in which the subtraction value becomes a positive value from a negative value as a starting point in the one pulse wave. FIG. 8B illustrates a pulse wave of a living body according to the subtraction value, and FIG. 8A illustrates a part of a pulse wave of a living body, which is ideal and expanded.

Furthermore, the CPU 31 extracts a feature point in the one pulse wave based on the subtraction value. For example, the CPU 31 extracts a largest value, a least value, a maximum value, a minimum value, or an inflection point as a feature point in each pulse wave. The CPU 31 calculates a value and time of the feature point from a waveform of the subtraction value. For example, the CPU 31 calculates the feature point by obtaining a speed pulse wave performing differentiation on a pulse wave or by obtaining an acceleration pulse wave performing differentiation twice on a pulse wave.

In FIG. 8A, a first peak (the maximum value) becomes a systolic peak and a second peak (the relative maximum value) becomes a reflective peak, in the period of one cycle. Furthermore, the minimum value after the second peak may be a notch representing a boundary between a systolic and a diastolic. A time period from a starting point in the period of one cycle to the systolic peak may represent a rising time (S. Time). A time period from a starting point in the period of one cycle to the reflective peak may represent a reflective time (R. Time). A time period from a starting point in the period of one cycle to the notch may represent a notch time. Furthermore, the CPU 31 may extract a least value of the period of one cycle as the feature point. In this manner, the CPU 31 calculates values and times of a plurality of feature points. Moreover, a largest value or a least value may be corrected based on the subtraction value as the notch according to an exemplary embodiment.

The CPU 31 calculates a feature amount from values and times of feature points included in one pulse wave. The feature amount in the specification refers to a value for calculating the blood pressures (SBP and DBP), and a value calculated from values and times of feature points in one pulse wave. It is possible to calculate the feature amount according to a predetermined formula.

Furthermore, the CPU 31 converts the feature amount into a blood pressure. The CPU 31 may convert the feature amount into a blood pressure value by using a regression line. FIG. 9A illustrates a graph illustrating calculating of BP_MAX along the right-side, and FIG. 9B illustrates a graph illustrating calculating of BP_MIN. In an exemplary embodiment, as illustrated in FIGS. 9A and 9B, two regression lines are stored in the memory 32 to calculate SBP (BP_MAX) and DBP (BP_MIN). In addition, the CPU 31 calculates each feature amount for SBP and DBP based on one pulse wave. Moreover, the CPU 31 respectively calculates SBP and DBP from two of the feature amounts by using the regression lines. Accordingly, the processor 30 calculates the blood pressure and outputs a signal representing the blood pressure to the display 40.

In addition, the regression lines are set by using a plurality of measurement results obtained in advance. For example, the biological information acquisition apparatus according to an exemplary embodiment may use a feature amount, and a cuff-type sphygmomanometer measures a blood pressure value simultaneously with respect to a plurality of measuring objects. Therefore, a database corresponding to the feature amount and the blood pressure value is constructed. Furthermore, regression analysis is performed with respect to data stored in the database, and the regression lines are obtained.

In this case, the regression lines may be distinguished by sex and age. For example, the regression lines may be set corresponding to sex and age such as men in their 20's, women in their 20's, men in their 30′, and women in their 30's. That is, the database may be constructed by obtaining data according to sex and age.

Meanwhile, the CPU 31 may convert a feature amount into a blood pressure by using a regression curve using a polynomial of the second degree or higher without being limited to the regression lines.

Furthermore, the CPU 31 may calculate a blood pressure based on a plurality of pulse waves. For example, the CPU 31 extracts feature points corresponding to respective n pulse waves (wherein n is an integer equal to 2 or greater) and calculates feature amounts. Therefore, n feature amounts are calculated as each feature amount is calculated for each pulse wave. In addition, the CPU 31 converts each of the n feature amounts into a blood pressure (SBP or DBP) by using regression lines. Therefore, n blood pressure values are calculated. In addition, an average value of n blood pressure values may be determined as the blood pressure. In this manner, measurement accuracy may be improved by calculating the feature amounts based on a plurality of the pulse waves.

Meanwhile, the blood pressure may be determined by excluding largest values or least values of n blood pressure values. For example, an average value of n−2 blood pressure values excluding largest values or least values among n blood pressure values may be determined as the blood pressure. Therefore, measurement accuracy may be further improved.

Meanwhile, one pulse wave (a period of one cycle) that cannot extract a feature point may be excluded from the calculation of the blood pressure. For example, when a maximum value and a minimum value used for calculating a feature amount are buried in noise and cannot be extracted due to an affection of the noise, the feature amount may not be calculated with respect to the period. Therefore, the blood pressure with respect to the one pulse wave (the period of one cycle), which cannot extract the feature point, may not be converted. Therefore, measurement accuracy of the blood pressure may be improved.

In this manner, the CPU 31 estimates a tendency such as rising and falling of the pulse wave based on the subtraction value and further estimates the maximum value, the least value, the relative maximum value, the minimum value, and the inflection point as the feature point. Furthermore, the CPU 31 calculates a feature amount from a plurality of feature points in each pulse wave and converts the feature amount into a blood pressure value by using regression lines which are obtained by a database in advance.

Referring again to FIG. 4, in operation S4, the display 40 displays a blood pressure represented by a signal input from the processor 30.

In an exemplary embodiment, the pulse wave of the living body according to a subtraction value resulting from subtracting a detection signal of a reflected light or a transmitted light near a dermis generating a large amount of noise, which is obtained by irradiating the living body with light of the second wavelength, from a detection signal of a reflected light or a transmitted light of a blood vessel, which is obtained by irradiating the living body with light of the first wavelength, is obtained. Therefore, it is possible to measure the pulse wave and the blood pressure of the living body with high accuracy due to the detection signal with less noise.

An exemplary embodiment describes another sensor.

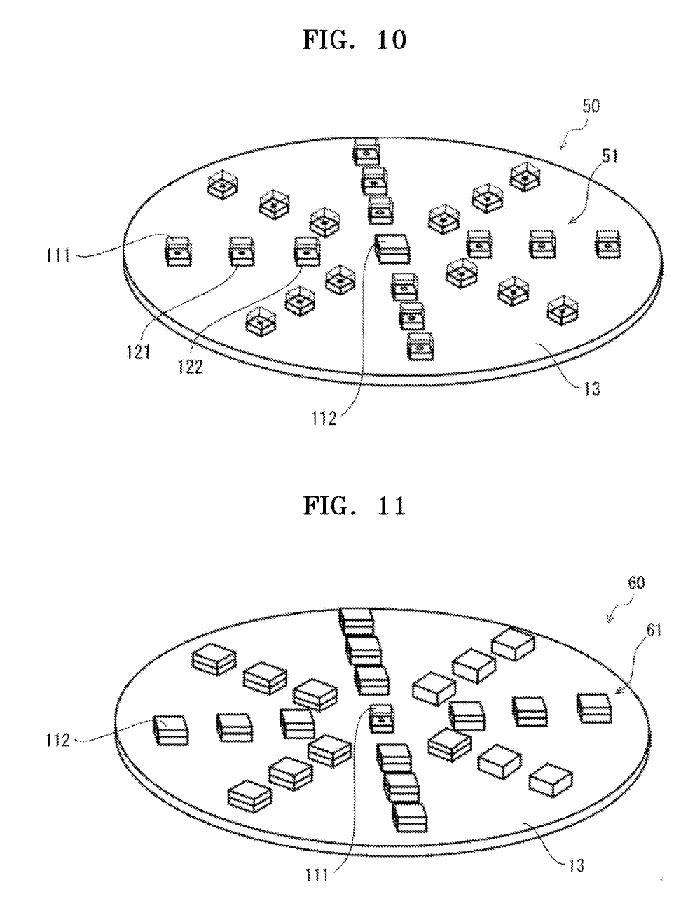

FIG. 10 is a schematic view of a sensor 50 according to an exemplary embodiment. While explaining the sensor 50 according to an exemplary embodiment, an element the same as that of the biological information acquisition apparatus 1 of the above exemplary embodiments is denoted using the same reference numeral, and repeated descriptions of the above exemplary embodiments are omitted.

As illustrated in FIG. 10, the sensor 50 according to an exemplary embodiment includes light source units 51 in which the first to third light sources 111 to 122 are arranged on an approximately straight line, which are arranged radially around the light receiver 112 on the substrate 13. Furthermore, the first to third light sources 111 to 122 of the light source units 51 are arranged on different concentric circles, respectively. In the specification, the concentric circles may include not just concentric circles in a strict sense but also a spiral and approximately concentric circles as will be understood by those of ordinary skill in the art.

That is, the first light source 111 of each of the light source units 51 may be arranged on a first circle around the light receiver 112. The second light source 121 of each of the light source units 51 may be arranged on a second circle around the light receiver 112, in which the second circle is smaller than the first circle. Furthermore, the third light source 122 of each of the light source units 51 may be arranged on a third circle around the light receiver 112, in which the third circle is smaller than the second circle.

A pulse wave to be measured has different waveforms according to a wearing state of the biological information acquisition apparatus with respect to a living body (for example, according to whether the biological information acquisition apparatus contacts the living body and to what degree of pressure). Therefore, the processor 30 selects one of the light source units 51 that satisfies a predetermined condition (for example, each of the first to third light sources 111 to 122 contacts the living body with a predetermined pressure) from the light source units 51, and obtains a pulse wave of the living body according to a detection signal obtained by irradiating the living body with a detection light from each of the first to third light sources 111 to 122 of the selected light source unit. Thus, the pulse wave may be measured with high accuracy.

Meanwhile, the sensor 50 according to an exemplary embodiment is constituted on the assumption that a detection signal obtained by irradiating the living body with a green light as a detection light is subtracted from a detection signal obtained by irradiating the living body with a red or infrared light as a detection light. However, all light sources on the sensor 50 may be a first light source 111 if noise is not removed by the detection signal obtained by irradiating the living body with the green light as the detection light. Here, intervals of the first light source adjacent in a radial direction and intervals of the first light source adjacent in a circumferential direction are suitably set based on a wavelength of an emitted light.

In this case, a pulse wave of the living body is obtained according to a detection signal having optimum characteristics (for example, ease of extracting a feature point) selected from sampled detection signals obtained by irradiating the living body with detection lights from all of the first light sources 111. Thus, the pulse wave of the living body may be measured with high accuracy. That is, one of the first light sources 111 capable of obtaining a detection signal having optimum characteristics among the plurality of the first light sources 111 arranged on a straight line and the same circle, and obtains a pulse wave of the living body according to a detection signal obtained by irradiating the living body with a detection light from the first light source 111.

Meanwhile, when data is collected by being distinguished according to sex, age and weight and by keeping distances between the first light sources 111 and the light receiver 112 constant, thus obtaining the database as described above, the distances from the first light sources 111 arranged on the same circle to the light receiver 112 are approximately the same when the first light sources 111 are arranged on the approximately concentric circle as described above. Therefore, an error of a blood pressure estimation algorithm by the database may be prevented from occurring.

Moreover, the number of the first light sources 111 may be more reduced compared to a case of arranging the first light sources 111 in a matrix shape on the substrate 13, by arranging the first light sources 111 on the approximately concentric circle as described above. As a result, the arrangement may contribute to weight reduction of the biological information acquisition apparatus.

Incidentally, U.S. Pat. No. 2,766,317 discloses a configuration of arranging light-emitting elements on a circle around a light receiving element. However, U.S. Pat. No. 2,766,317 is related to a measurement of oxygen saturation of blood.

A sensor from which noise is not removed by the detection signal obtained by irradiating the living body with a green light as a detection light will now be described according to another exemplary embodiment.

FIG. 11 is a schematic view of a sensor 60 according to another exemplary embodiment. An element the same as that of the biological information acquisition apparatus 1 of the above exemplary embodiments in the sensor 60 according to an exemplary embodiment is denoted using the same reference numeral and repeated descriptions of the above exemplary embodiments are omitted.

As illustrated in FIG. 11, the sensor 60 according to an exemplary embodiment includes light-receiving units 61 in which the plurality of light receivers 112 are arranged on an approximately straight line, which is arranged radially around the first light source 111 on the substrate 13. Furthermore, the light receivers 112 of each of the light-receiving units 61 are arranged on different concentric circles, respectively. Here, intervals of the light receivers 112 adjacent in a radial direction and intervals of the light receivers 112 adjacent in a circumferential direction are suitably set based on a wavelength of an emitted light of the first light source 111.

As described above, a pulse wave to be measured has different waveforms according to a wearing state of the biological information acquisition apparatus with respect to a living body. Therefore, one of the light receivers 112, which satisfies a predetermined condition (for example, contacting the living body with a predetermined pressure), is selected from the light receivers 112, and a pulse wave of the living body is obtained according to a detection signal of the selected light receiver 112. Thus, measurement accuracy of the pulse wave may be improved.

Meanwhile, data may be collected by being distinguished according to sex, age and weight and by keeping distances between the first light source 111 and the light receivers 112 constant, thus obtaining the database as described above. When the light receivers 112 are arranged on the same circle as described above, the distances from the light receivers 112 arranged on the same circle to the first light source 111 are approximately constant. Therefore, an error of a blood pressure estimation algorithm by the database may be prevented from occurring.

Meanwhile, the number of the light receivers 112 may be more reduced compared to a case of arranging the light receivers 112 in a matrix shape on the substrate 13, by arranging the light receivers 112 on the approximately concentric circle as described above. As a result, the arrangement may contribute to weight reduction of the biological information acquisition apparatus.

A sensor from which noise is not removed by the detection signal obtained by irradiating the living body with a green light as a detection light will now be described according to another exemplary embodiment.

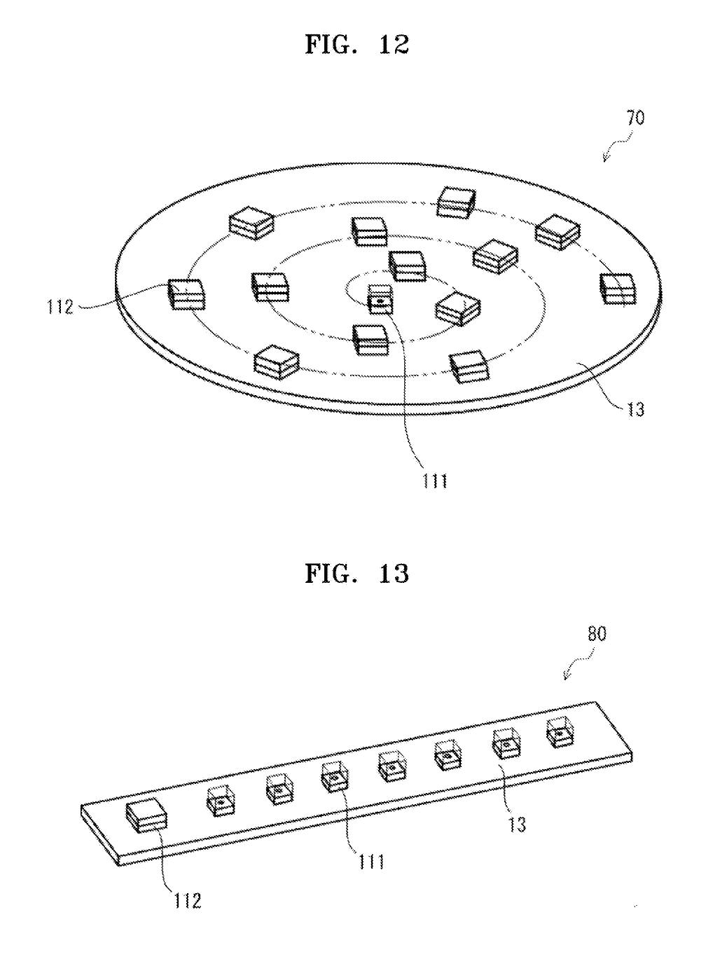

FIG. 12 is a schematic view of a sensor 70 according to another exemplary embodiment. An element the same as that of the biological information acquisition apparatus 1 of the above exemplary embodiments in the sensor 70 according to an exemplary embodiment is denoted using the same reference numeral and repeated descriptions of the above exemplary embodiments are omitted.

As illustrated in FIG. 12, the sensor 70 according to an exemplary embodiment includes the substrate 13 on which the light receivers 112 are arranged on a spiral (illustrated by a two-dot chain line in FIG. 12) around the first light source 111. Also in this case, one of the light receivers 112, which satisfies a predetermined condition, is selected from the light receivers 112 and a pulse wave of the living body is obtained according to a detection signal of the selected light receiver 112. Thus, measurement accuracy of the pulse wave may be improved. Here, intervals of the light receivers 112 adjacent to each other on the spiral may be suitably set based on a wavelength of an emitted light of the first light source 111.

Moreover, according to an exemplary embodiment, the light receivers 112 are arranged on the spiral around the first light source 111. However, it is possible to arrange the first light sources 111 on the spiral around the light receiver 112 and select one of the first light sources 111, and thus a pulse wave of the living body may be obtained according to a detection signal of a reflected light obtained by irradiating the living body with a detection light from the selected first light source 111.

A sensor from which noise is not removed by the detection signal obtained by irradiating the living body with a green light as a detection light will now be described according to another exemplary embodiment.

FIG. 13 is a schematic view of a sensor 80 according to another exemplary embodiment. An element the same as that of the biological information acquisition apparatus 1 of the above exemplary embodiments in the sensor 80 according to an exemplary embodiment is denoted using the same reference numeral and repeated descriptions of the above exemplary embodiments are omitted.

As illustrated in FIG. 13, the sensor 80 according to an exemplary embodiment includes the substrate 13 on which the plurality of first light sources 111 and the light receiver 112 are arranged on an approximately straight line. Also in this case, one of the first light sources 111, which satisfies a predetermined condition, is selected from the first light sources 111, and a pulse wave of the living body is obtained according to a detection signal of the selected first light source 111. Thus, measurement accuracy of the pulse wave may be improved.

Meanwhile, according to an exemplary embodiment, the plurality of first light sources 111 and the light receiver 112 are arranged on the approximately straight line. However, it is possible to arrange the first light source 111 and a plurality of the light receivers 112 on the approximately straight line, and thus a pulse wave of the living body may be obtained based on a detection signal obtained by irradiating the living body with a detection light from the selected light receiver 112.

A sensor from which noise is not removed by the detection signal obtained by irradiating the living body with a green light as a detection light will now be described according to another exemplary embodiment.

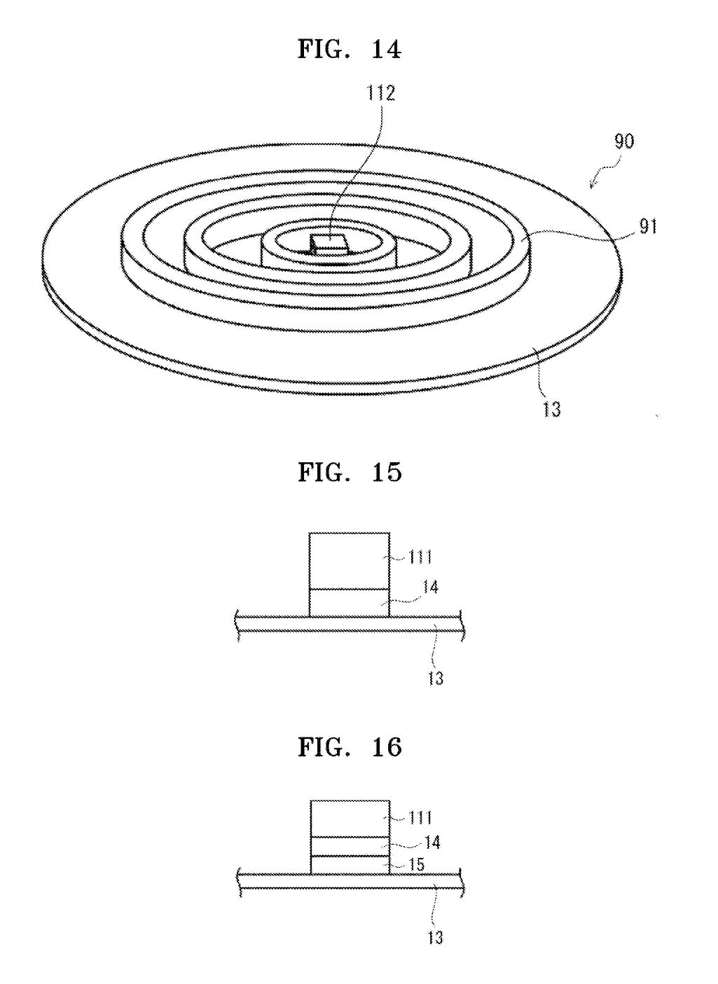

FIG. 14 is a schematic view of a sensor 90 according to another exemplary embodiment. An element the same as that of the biological information acquisition apparatus 1 of the above exemplary embodiments in the sensor 90 according to an exemplary embodiment is denoted using the same reference numeral and repeated descriptions of the above exemplary embodiments are omitted.

As illustrated in FIG. 14, the sensor 90 according to an exemplary embodiment includes a plurality of ring-shaped light sources 91 with different diameters arranged around the light receiver 112 on the substrate 13. Also in this case, one of the light sources 91, which satisfies a predetermined condition (for example, contacting the living body with a predetermined pressure), is selected from the light sources 91, and a pulse wave of the living body is obtained according to a detection signal obtained by irradiating the living body with a detection light from the selected light sources 91. Thus, measurement accuracy of the pulse wave may be improved. Here, intervals of the light sources 91 adjacent to each other may be suitably set based on wavelengths of emitted lights of the light sources 91.

Moreover, according to an exemplary embodiment, the ring-shaped light sources 91 are arranged around the light receiver 112 on the substrate 13. However, it is possible to arrange a plurality of ring-shaped light receivers around a point light source and select one of the light receivers satisfying a predetermined condition, and thus a pulse wave of the living body may be obtained according to a detection signal of the selected light receiver.

In another exemplary embodiment, the substrate 13 may be a flexible substrate. Therefore, a light source and a light receiver may appropriately contact a living body as the substrate 13 is bent along the living body, for example, a curved wrist.

When a biological information acquisition apparatus is attached onto the living body, in which the substrate 13 is a flexible substrate, the substrate 13 is bent and may transform the concentric circles described above. That is, in a state of the biological information acquisition apparatus that is attached onto the living body, one of the light source and light receiver may be arranged on approximately concentric ellipsoids centering around one of the other light source and light receiver in a state of flattening the substrate 13 to arrange the light source or the light receiver on an approximately concentric circle.

As described above, it is possible to adopt a configuration as below when a contact pressure of a light source or a light receiver on a living body is detected.

FIG. 15 is a schematic view of a sensor configured to be able to detect a contact pressure of a first light source on a living body according to another exemplary embodiment.

As illustrated in FIG. 15, a sensor according to an exemplary embodiment includes a pressure detector 14 between the first light source 111 and the substrate 13. The pressure detector 14 may use a pressure sensor.

When the above configuration is adopted with respect to, for example, a sensor including the plurality of the first light sources 111, a processor selects one of the first light source 111 having at least a predetermined pressure value or more from the first light sources 111 based on a detection signal of the pressure detector 14, and obtains a pulse wave of the living body according to a detection signal obtained by irradiating the living body with a detection light from the selected first light source 111. Thus, it is possible to avoid a difficulty in measuring the pulse wave of the living body due to variations in an attaching state of the biological information acquisition apparatus to the living body or individual differences of the living body.

Meanwhile, FIG. 15 illustrates a configuration capable of detecting the contact pressure of the first light sources 111 on the living body. However, for example, a sensor including the plurality of light receivers 112 may dispose the pressure detector 14 between each of the light receivers 112 and the substrate 13.

According to another exemplary embodiment, a sensor has a configuration capable of adjusting a contact pressure of a light source or a light receiver on a living body to a predetermined value.

FIG. 16 is a schematic view of a configuration capable of adjusting a contact pressure of a first light source on a living body to a predetermined value according to another exemplary embodiment.

As illustrated in FIG. 16, according to an exemplary embodiment, the pressure detector 14 and a pressure adjuster 15 are disposed between the first light source 111 and the substrate 13. The pressure adjuster 15 may use an actuator.

When the above configuration is adopted with respect to, for example, a sensor including the plurality of the first light sources 111, a processor controls the signal pressure adjuster 15 based on a detection signal from the pressure detector 14, and adjusts a contact pressure of the first light sources 111 on the living body to a predetermined value. Furthermore, a pulse wave of the living body is obtained according to a detection signal having optimum characteristics (for example, easy to extract a feature point) obtained by irradiating the living body with a detection light from the first light sources 111. Thus, it is possible to avoid a difficulty in measuring the pulse wave of the living body due to variations in an attaching state of the biological information acquisition apparatus to the living body or individual differences of the living body.

Meanwhile, FIG. 16 illustrates a configuration capable of detecting the contact pressure of the first light sources 111 on the living body. However, for example, a sensor including the plurality of light receivers 112 may dispose the pressure detector 14 and the pressure adjuster 15 between each of the light receivers 112 and the substrate 13.

According to another exemplary embodiment, when a sensor including the plurality of first light sources 111 or the plurality of light receivers 112 are used, a blood pressure of a living body is calculated using a pulse wave transit time (PWTT) method based on a pulse wave obtained from a different position of the living body by operating the first light sources 111 or the light receivers 112 having different distances from one of the other first light sources 111 in the center and the other light receivers 112.

According to another exemplary embodiment, when a sensor includes the plurality of first light sources 111, frequencies of emitted lights from the first light sources 111 may be changed according to distances of the plurality of light receivers 112. For example, the frequencies of the emitted lights from the first light sources 111 increases as the sensor gets farther away from the light receivers 112. Therefore, it is possible to adjust a degree of penetration of the emitted lights from the first light sources 111 to the living body.

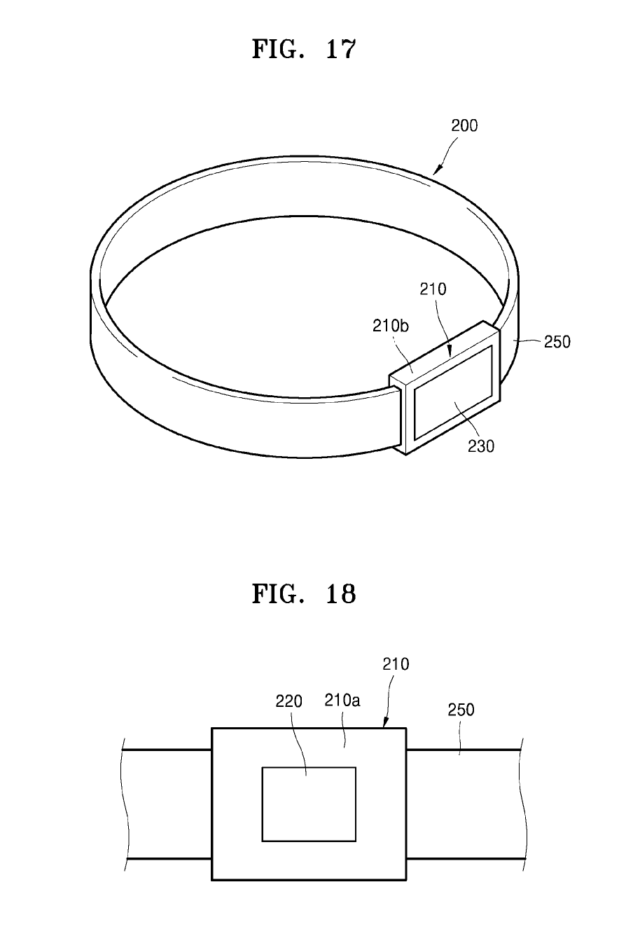

FIG. 17 is a schematic view of a wrist watch-type terminal 200 according to another exemplary embodiment, and FIG. 18 is a view of a back of the wrist watch-type terminal of FIG. 17. As illustrated in FIGS. 17 and 18, the wrist watch-type terminal 200 includes a main body 210, a sensor 220, and a display 230 arranged in a front surface 210b of the main body 210. The sensor 220 is arranged on a rear surface 210a of the main body 210 to contact a wrist of a living body. The sensor 220 may be sensors 10, 50, 60, 70, 80 and 90 according to the above exemplary embodiments. The main body 210 is attached onto the wrist of the living body by a band 250, and thus the sensor 220 contacts skin on the wrist. The AFE 20 (of FIG. 1) and the processor 30 (of FIG. 1) may be arranged in the main body 210. The display 230 may display blood pressures (SBP and DBP). Furthermore, the display 230 may display time independently or display time along with a blood pressure.

For example, in the above exemplary embodiments, a pulse wave of a living body is measured according to a reflected light that is a detection light reflected in a living body. However, it is possible to measure the pulse wave of the living body according to a transmitted light which is a detection light being transmitted in a living body. In this case, a light source and a light receiver may be arranged so that the living body is located therebetween.

For example, in some of the above exemplary embodiments, a sensor including the plurality of the first light sources 111 or light receivers 112 is described but the sensor is not limited thereto. That is, the first light sources 111 or the light receivers 112 may be arranged on the substrate 13 regularly or irregularly.

For example, it is possible to dispose the biological information acquisition apparatus according to the above exemplary embodiment in a wrist band or a wearable terminal capable of contacting a living body other than the wrist watch-type terminal. Furthermore, the wearable terminal includes a wireless communicator for communicating with the sensors 10, 50, 60, 70, 80 and 90. However, other configurations (for example, the processor 30, the display 40, and so on) may be included in a smart phone. In this case, analog data or digital data obtained by the wrist watch-type terminal is transmitted to the smart phone by the wireless communicator. Furthermore, the smart phone having the data may perform all or part of the process for calculating a blood pressure.

In addition, the exemplary embodiments may also be implemented through computer-readable code and/or instructions on a medium, e.g., a computer-readable medium, to control at least one processing element to implement any above-described embodiments. The medium may correspond to any medium or media which may serve as a storage and/or perform transmission of the computer-readable code.

The computer-readable code may be recorded and/or transferred on a medium in a variety of ways, and examples of the medium include recording media, such as magnetic storage media (e.g., ROM, floppy disks, hard disks, etc.) and optical recording media (e.g., compact disc read only memories (CD-ROMs) or digital versatile discs (DVDs)), and transmission media such as Internet transmission media. Thus, the medium may have a structure suitable for storing or carrying a signal or information, such as a device carrying a bitstream according to one or more exemplary embodiments. The medium may also be on a distributed network, so that the computer-readable code is stored and/or transferred on the medium and executed in a distributed fashion. Furthermore, the processing element may include a processor or a computer processor, and the processing element may be distributed and/or included in a single device.

The foregoing exemplary embodiments are examples and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for acquiring biological information, the apparatus comprising:
a first detector configured to:
irradiate a living body with a first light of a first wavelength; and
detect a second light that is reflected from or transmitted in the living body by irradiating the first light of the first wavelength;
a second detector configured to:
irradiate the living body with a third light of a second wavelength; and
detect a fourth light that is reflected from or transmitted in the living body by irradiating the third light of the second wavelength; and
a processor configured to determine a pulse wave of the living body based on a subtraction value of subtracting a detection signal that is obtained by detecting the fourth light from a detection signal that is obtained by detecting the second light.

2. The apparatus of claim 1, wherein the first light of the first wavelength is a red or infrared light.

3. The apparatus of claim 1, wherein the third light of the second wavelength is a green light.

4. The apparatus of claim 1, wherein the first light of the first wavelength is scattered in a blood vessel disposed deeper than a dermis of the living body, and
the third light of the second wavelength is scattered near the dermis.

5. The apparatus of claim 1, wherein the first detector comprises a first light source configured to emit the first light of the first wavelength, and a first light receiver configured to detect the second light reflected from or transmitted in the living body,
the second detector comprises a second light source configured to emit the third light of the second wavelength, and a second light receiver configured to detect the fourth light reflected from or transmitted in the living body, and
a distance between the first light source and the first light receiver is farther than a distance between the second light source and the second light receiver.

6. The apparatus of claim 5, wherein the second light receiver is shared with the first light receiver.

7. The apparatus of claim 1, wherein the irradiation of the living body with the first light of the first wavelength and the irradiation of the living body with the third light of the second wavelength are performed during a period so that irradiation timings thereof are not overlapped.

8. The apparatus of claim 7, wherein the period is substantially equal to a period of the pulse wave of the living body.

9. A wrist watch-type terminal comprising a biological information acquisition apparatus, the biological information acquisition apparatus comprising:
a first detector configured to:
irradiate a living body with a first light of a first wavelength; and
detect a second light that is reflected from or transmitted in the living body by irradiating the first light of the first wavelength;
a second detector configured to:
irradiate the living body with a third light of a second wavelength; and
detect a fourth light that is reflected from or transmitted in the living body by irradiating the third light of the second wavelength; and
a processor configured to determine a pulse wave of the living body based on a subtraction value of subtracting a detection signal that is obtained by detecting the fourth light from a detection signal that is obtained by detecting the second light.

10. A method of acquiring biological information, the method comprising:
irradiating a living body with a first light of a first wavelength;
detecting a second light that is reflected from or transmitted in the living body by irradiating the first light of the first wavelength;
irradiating the living body with a third light of a second wavelength;
detecting a fourth light that is reflected from or transmitted in the living body by irradiating the third light of the second wavelength; and
determining a pulse wave of the living body based on a subtraction value of subtracting a detection signal that is obtained by detecting the fourth light from a detection signal that is obtained by detecting the second light.

11. The method of claim 10, wherein the first light of the first wavelength is a red or infrared light.

12. The method of claim 10, wherein the third light of the second wavelength is a green light.

13. The method of claim 10, wherein the first light of the first wavelength is scattered in a blood vessel disposed deeper than a dermis of the living body, and
the third light of the second wavelength is scattered near the dermis.

14. The method of claim 10, wherein the irradiation of the living body with the first light of the first wavelength and the irradiation of the living body with the third light of the second wavelength are performed during a period so that irradiation timings thereof are not overlapped.

15. The method of claim 14, wherein the period is substantially equal to a period of the pulse wave of the living body.

16. An apparatus for acquiring biological information, the apparatus comprising:
a substrate;
a light source disposed on the substrate, the light source being configured to emit a light to a living body;
a light receiver disposed on the substrate, the light receiver being configured to receive a light that is reflected from or transmitted in the living body; and
a processor configured to determine a pulse wave of the living body based on a detection signal of the received light,
wherein one of the light source and the light receiver is disposed around another one of the light source and the light receiver.

17. The apparatus of claim 16, wherein the one of the light source and the light receiver respectively comprises light sources or light receivers disposed on two or more approximately concentric circles, and
the processor is further configured to:
select one of the light sources or the light receivers satisfying a condition; and
determine the detection signal of the light that is received by a light receiver corresponding to the selected one of the light sources or the light receivers.

18. The apparatus of claim 16, wherein the one of the light source and the light receiver respectively comprises light sources or light receivers disposed on an approximately straight line, and
the processor is further configured to:
select one of the light sources or the light receivers satisfying a condition; and
determine the detection signal of the light that is received by a light receiver corresponding to the selected one of the light sources or the light receivers.

19. The apparatus of claim 16, wherein the one of the light source and the light receiver respectively comprises light sources or light receivers disposed on a spiral centering around the other one of the light source and the light receiver and
the processor is further configured to:
select one of the light sources or the light receivers satisfying a condition; and
determine the detection signal of the light that is received by a light receiver corresponding to the selected one of the light sources or the light receivers.

20. The apparatus of claim 16, wherein the one of the light source and the light receiver respectively comprises light sources or light receivers that are ring-shaped, and
the processor is further configured to:
select one of the light sources or the light receivers satisfying a condition; and
determine the detection signal of the light that is received by a light receiver corresponding to the selected one of the light sources or the light receivers.

21. The apparatus of claim 16, further comprising:
a pressure detector configured to detect a contact pressure of the light source or the light receiver on the living body,
wherein the processor is further configured to:
select the light source or the light receiver contacting the living body with the contact pressure corresponding to a pressure; and
determine the detection signal of the light that is received by a light receiver corresponding to the selected light source or light receiver.

22. The apparatus of claim 16, further comprising:
a pressure detector configured to detect a contact pressure of the light source or the light receiver on the living body, the light source or the light receiver being disposed on an approximately concentric circle; and
a pressure adjuster configured to adjust the contact pressure on the living body to a pressure.

23. The apparatus of claim 16, wherein the processor is further configured to:
determine a pulse wave in a different position of the living body by operating the other one of the light source and the light receiver at a different distance from the one of the light source and the light receiver; and
determine a blood pressure of the living body based on a propagation time of the determined pulse wave.

24. The apparatus of claim 16, wherein a wavelength of the light emitted to the living body changes based on a distance between the light source and the light receiver that respectively are on different sides.

25. The apparatus of claim 16, wherein the substrate is flexible.

26. The apparatus of claim 25, wherein, in response to a state of bending the flexible substrate as the biological information acquisition apparatus is attached onto the living body, the one of the light source and the light receiver is disposed on an approximately concentric ellipsoid centering around the other one of the light source and the light receiver, and
in response to a state of flattening the flexible substrate, the one of the light source and the light receiver is disposed on an approximately concentric circle centering around the other one of the light source and the light receiver.

27. The apparatus of claim 16, wherein the light source comprises:
a first light source configured to emit a light of a first wavelength; and
a second light source configured to emit a light of a second wavelength, and
the processor is further configured to determine the pulse wave based on a subtraction value of subtracting a detection signal that is determined by irradiating the light of the second wavelength from a detection signal that is determined by irradiating the light of the first wavelength.

28. A wrist watch-type terminal comprising a biological information acquisition apparatus, the biological information acquisition apparatus comprising:
a substrate;
a light source disposed on the substrate, the light source being configured to emit a light to a living body;
a light receiver disposed on the substrate, the light receiver being configured to receive a light that is reflected from or transmitted in the living body; and
a processor configured to determine a pulse wave of the living body based on a detection signal of the received light,
wherein one of the light source and the light receiver is disposed around another one of the light source and the light receiver.
